(19) United States
(12) Patent Application Publication
(10) Pub. No.: US 2024/0264192 A1
(43) Pub. Date: Aug. 8, 2024

(54) AUTOMATIC ANALYZING APPARATUS AND CONTROL METHOD THEREOF

(71) Applicant: CANON MEDICAL SYSTEMS CORPORATION, Otawara-shi (JP)

(72) Inventors: Tetsushi YAMAMOTO, Otawara (JP); Tatsuya MURATA, Otawara (JP); Tomio NABATAME, Otawara (JP); Kenji YAMASAKI, Otawara (JP); Tomomi MURAMATSU, Otawara (JP)

(73) Assignee: CANON MEDICAL SYSTEMS CORPORATION, Otawara-shi (JP)

(21) Appl. No.: 18/428,022

(22) Filed: Jan. 31, 2024

(30) Foreign Application Priority Data
Feb. 2, 2023 (JP) 2023-014890

(51) Int. Cl. G01N 35/10; G01N 35/02; G06V 20/52

(52) U.S. Cl. CPC G01N 35/1011; G01N 35/026; G01N 35/1079; G01N 2035/1025; G06V 20/52

(57) ABSTRACT

An automatic analyzing apparatus according to the present embodiment comprises a first imager configured to image a sample container in which a sample is contained and whose upper surface is sealed with a cap; and processing circuitry configured to acquire a liquid level height of the sample contained in the sample container based on first image data of the sample container imaged by the first imager; and determine, based on the liquid level height, a first parameter related to a descent operation of a piercing arm, and controls, based on the first parameter, the descent operation of the piercing arm, and the piercing arm holding a piercer needle for piercing the cap of the sample container.

### CROSS REFERENCE TO RELATED APPLICATIONS

This application is based upon and claims the benefit of priority from the prior Japanese Patent Application No. 2023-014890, filed on Feb. 2, 2023, the entire contents of which are incorporated herein by reference.

### FIELD

Embodiments described in the present specification and drawings relate to an automatic analyzing apparatus and a control method thereof.

### BACKGROUND

An automatic analyzing apparatus adds reagents corresponding to various test items to a sample such as blood of a patient containing a component to be analyzed. Each reagent reacts with a specific component of the sample. The automatic analyzing apparatus analyzes a component of the sample corresponding to the test item by optically measuring this reaction, for example.

For analyzing this sample such as blood, the sample such as blood is contained in a sample container whose upper surface is sealed with a cap. In an automatic analyzing apparatus, when an upper surface of a sample container is sealed with a cap, prior to insertion of a sampling probe for sucking a sample into the sample container, a piercing arm holding a piercer needle is lowered from above the sample container to perform a descent operation for piercing the cap of the sample container. In the descent operation of the piercing arm, the piercing arm is lowered based on a preset descending amount of the piercing arm, and the cap is pierced by the piercer needle. The descending amount of the piercing arm is set for each type of the sample container and the cap so that the tip of the piercer needle pierces the cap of the sample container and descends to a position where the tip of the piercer needle does not touch the sample.

However, since there are many types of sample containers and caps, there may be a sample container and a cap in which the descending amount of the piercing arm is not set. In this case, in the automatic analyzing apparatus, in a case where it is not possible to use the sample container and the cap in which the descending amount of the piercing arm is not set, and a new sample container and cap are used, it is necessary to set the descending amount of the piercing arm with respect to the sample container and the cap, therefore the work load of the user is large.

### BRIEF DESCRIPTION OF THE DRAWINGS

FIG. 1 is a block diagram illustrating an example of a functional configuration of an automatic analyzing apparatus according to a first embodiment;

FIG. 2 is a diagram illustrating an example of a configuration of an analysis mechanism in the automatic analyzing apparatus illustrated in FIG. 1;

FIGS. 3A and 3B are perspectives view illustrating an example of a configuration of a piercing arm and a piercing drive mechanism included in the analysis mechanism illustrated in FIG. 2;

FIG. 4 is a diagram illustrating an example of a configuration of a piercer needle included in the piercing arm illustrated in FIGS. 3A and 3B;

FIG. 5 is a schematic view illustrating movement trajectories of a sampling probe and a piercer needle in the automatic analyzing apparatus illustrated in FIG. 1;

FIG. 6 is a flowchart diagram for explaining the content of a descent operation control process executed by the automatic analyzing apparatus illustrated in FIG. 1;

FIG. 7 is a diagram conceptually illustrating an example of a layout of a sample container on a sample rack and a first imager in the automatic analyzing apparatus according to the first embodiment;

FIGS. 8A to 8C are views for explaining how the piercer needle and the sampling probe descend in order to perform a piercing operation by the piercer needle and a suction operation by the sampling probe;

FIG. 9 is a diagram illustrating an example of a layout of a rack sampler in an automatic analyzing apparatus according to a second embodiment;

FIG. 10 is a block diagram illustrating an example of a functional configuration of an automatic analyzing apparatus according to a third embodiment;

FIG. 11 is a flowchart diagram for explaining the content of a descent operation control process executed by the automatic analyzing apparatus illustrated in FIG. 10;

FIG. 12 is a diagram conceptually illustrating an example of a layout of a sample container on a sample rack, a first imager, and a second imager in the automatic analyzing apparatus according to the third embodiment;

FIG. 13 is a diagram illustrating an example of a configuration in a case where an external conveyance apparatus is connected to an automatic analyzing apparatus according to a fourth embodiment;

FIG. 14 is a diagram illustrating an example of a configuration of a disk sampler that stores a sample container in an automatic analyzing apparatus according to a fifth embodiment;

FIG. 15 is a block diagram illustrating an example of a functional configuration of an automatic analyzing apparatus according to a seventh embodiment;

FIG. 16 is a diagram for explaining the content of a descent operation control process executed by the automatic analyzing apparatus illustrated in FIG. 15;

FIG. 17 is a diagram conceptually illustrating an example of a layout of a sample container on a sample rack, a first imager, and a barcode reader in the automatic analyzing apparatus according to the seventh embodiment;

FIG. 18 is a block diagram illustrating an example of a functional configuration of an automatic analyzing apparatus according to an eighth embodiment;

FIG. 19 is a diagram for explaining the content of a descent operation control process executed by the automatic analyzing apparatus illustrated in FIG. 18;

FIG. 20 is a diagram illustrating an example of a configuration of a sample container whose entire surface is formed of a transparent portion in an automatic analyzing apparatus according to a modification; and

FIG. 21 is a diagram illustrating an example of a configuration of a sample container whose partial surface formed of a transparent portion in an automatic analyzing apparatus according to a modification.

### DETAILED DESCRIPTION

Hereinafter, respective embodiments of the automatic analyzing apparatus and control method thereof will be described with reference to the accompanying drawings. In the embodiments below, the same reference signs are given for identical components in terms of configuration and function, and duplicate description is omitted.

### First Embodiment

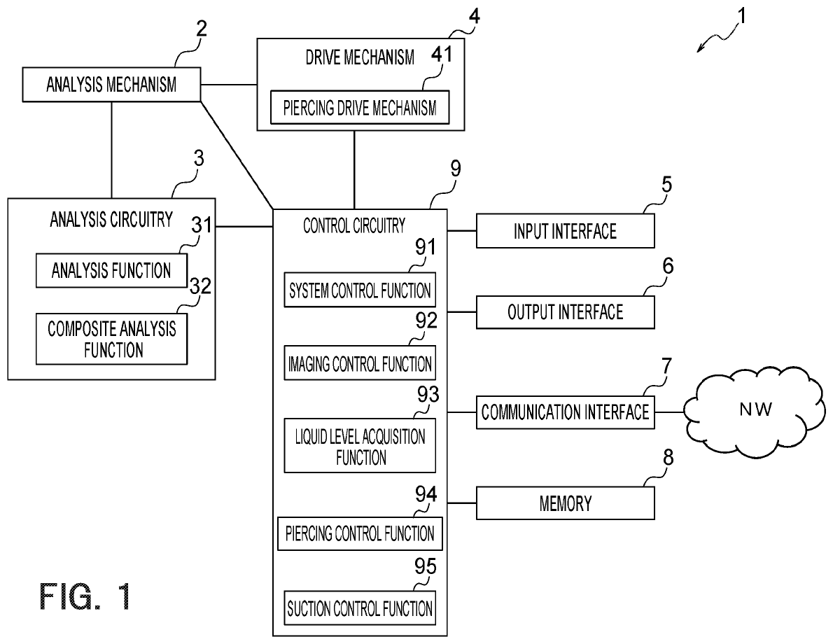

FIG. 1 is a block diagram illustrating an example of a functional configuration of an automatic analyzing apparatus 1 according to a first embodiment. In the present embodiment, the automatic analyzing apparatus 1 is, for example, a blood coagulation analyzing apparatus. In the following description, the present embodiment will be described by exemplifying a case where a sample collected from a subject is blood and the automatic analyzing apparatus 1 is a blood coagulation analyzing apparatus, but the present embodiment is also applicable to other types of samples such as urine and other types of automatic analyzing apparatuses such as automatic analyzing apparatuses that perform biochemical tests.

As illustrated in FIG. 1, an automatic analyzing apparatus 1 according to the present embodiment includes an analysis mechanism 2, an analysis circuitry 3, a drive mechanism 4, an input interface 5, an output interface 6, a communication interface 7, a memory 8, and a control circuitry 9.

The analysis mechanism 2 generates a mixed liquid obtained by mixing a blood specimen that is a specimen of a subject with a coagulation reagent that is a reagent used for each test item. In addition, depending on the test item, the analysis mechanism 2 mixes the standard liquid diluted at a predetermined ratio with the reagent used in the test item. The analysis mechanism 2 continuously measures an optical physical property value of the mixed liquid of the blood specimen and the reagent or the mixed liquid of the standard liquid and the reagent. By this measurement, for example, standard data represented by transmitted light intensity, absorbance, scattered light intensity, and the like, and test data are generated.

The analysis circuitry 3 is a processor that generates calibration data and analysis data related to coagulation of a blood specimen by analyzing standard data and test data generated by the analysis mechanism 2. For example, the analysis circuitry 3 reads an analysis program from the memory 8, and analyzes the standard data and the test data according to the read analysis program. The analysis circuitry 3 may include a storage area for storing at least part of the data stored in the memory 8.

The drive mechanism 4 drives the analysis mechanism 2 under the control of the control circuitry 9. The drive mechanism 4 is implemented by, for example, a gear, a stepping motor, a belt conveyor, a lead screw, and the like. In particular, the drive mechanism 4 includes a piercing drive mechanism 41 for driving a piercing arm to be described later.

The input interface 5 receives, for example, setting of an analysis parameter or the like of each test item related to the blood specimen requested to be measured from an operator or via an in-hospital network NW. The input interface 5 is implemented by, for example, a mouse, a keyboard, a touch pad to which an instruction is input by touching an operation surface, and the like. The input interface 5 is connected to the control circuitry 9, converts an operation instruction input from the operator into an electric signal, and outputs the electric signal to the control circuitry 9. Note that, in the present specification, the input interface 5 is not limited to one having physical operation components such as a mouse and a keyboard. For example, an electric signal processing circuitry that receives an electric signal corresponding to an operation instruction input from an external input device provided separately from the automatic analyzing apparatus 1 and outputs the electric signal to the control circuitry 9 is also included in the example of the input interface 5.

The output interface 6 is connected to the control circuitry 9 and outputs a signal supplied from the control circuitry 9. The output interface 6 is implemented by, for example, a display circuitry, a print circuitry, an audio device, and the like. Examples of the display circuitry include a CRT display, a liquid crystal display, an organic EL display, an LED display, and a plasma display. Note that the display circuitry also includes a processing circuitry that converts data representing a display target into a video signal and externally outputs the video signal. The printed circuitry includes, for example, a printer or the like. An output circuitry that externally outputs data representing the printing target is also included in the printed circuitry. The audio device includes, for example, a speaker or the like. Note that an output circuitry that externally outputs an audio signal is also included in the audio device.

The communication interface 7 is connected to, for example, the in-hospital network NW. The communication interface 7 performs data communication with a hospital information system (HIS) via the in-hospital network NW. Note that the communication interface 7 may perform data communication with the HIS via a laboratory information system (LIS) connected to the in-hospital network NW.

The memory 8 includes a magnetic or optical recording medium, a recording medium readable by a processor such as a semiconductor memory, or the like. Note that the memory 8 is not necessarily implemented by a single storage device. For example, the memory 8 may be implemented by a plurality of storage devices.

The memory 8 stores the analysis program executed by the analysis circuitry 3 and a control program for realizing functions included in the control circuitry 9. The memory 8 stores the calibration data generated by the analysis circuitry 3 for each test item. The memory 8 stores the analysis data generated by the analysis circuitry 3 for each blood specimen. The memory 8 stores a request for a test input from the operator or a request for a test received by the communication interface 7 via the in-hospital network NW.

The control circuitry 9 is a processor that functions as a center of the automatic analyzing apparatus 1. The control circuitry 9 is an example of a processing circuitry. The control circuitry 9 executes a program stored in the memory 8 to implement a function corresponding to the executed program. The control circuitry 9 may include a storage area for storing at least part of the data stored in the memory 8.

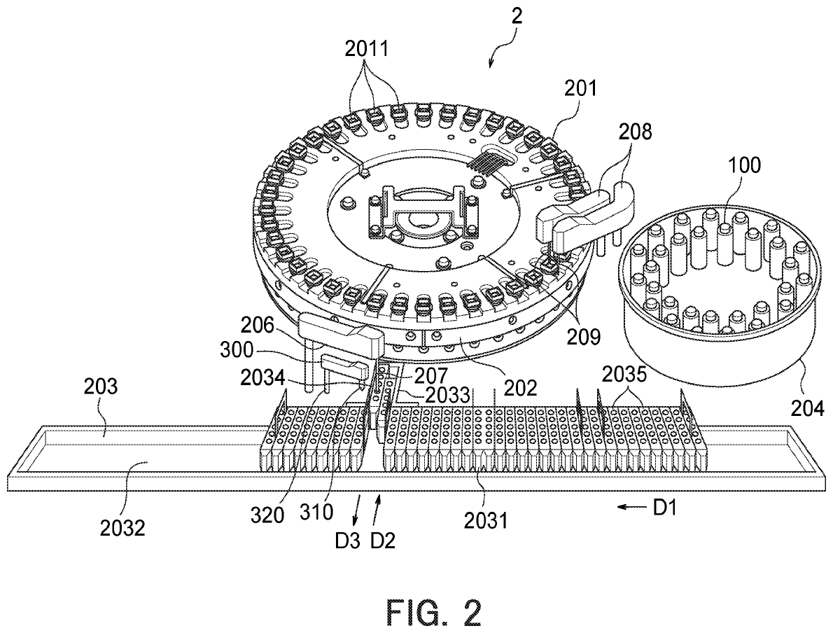

FIG. 2 is a diagram illustrating an example of a configuration of an analysis mechanism in the automatic analyzing apparatus 1 illustrated in FIG. 1. As illustrated in FIG. 2, the analysis mechanism 2 according to the present embodiment includes a reaction disk 201, a thermostatic unit 202, a rack sampler 203, a reagent storage 204, a sampling arm 206, a sampling probe 207, a reagent dispensing arm 208, and a reagent dispensing probe 209.

The reaction disk 201 holds a plurality of reaction containers (cuvettes) 2011 arranged in an annular shape. The reaction disk 201 conveys the reaction containers 2011 along a predetermined path. Specifically, during the analysis operation of the specimen, the reaction disk 201 is alternately rotated and stopped at predetermined time intervals by the drive mechanism 4. The reaction containers 2011 are made of, for example, polypropylene (PP) or acrylic.

The constant temperature unit 202 stores a heating medium set at a predetermined temperature, and immerses the reaction containers 2011 in the stored heating medium to raise the temperature of the mixed liquid contained in the reaction containers 2011.

The rack sampler 203 movably supports a sample rack 2031 capable of holding a plurality of sample containers 2035, and a blood specimen that is a specimen requested to be measured is contained in the plurality of sample containers 2035. In the example illustrated in FIG. 2, a sample rack 2031 capable of holding five tubes of sample containers 2035 in parallel is illustrated.

The rack sampler 203 is provided with a conveyance area 2032 for conveying the sample rack 2031. That is, the sample rack 2031 is conveyed from the loading position where the sample rack 2031 is loaded to the collection position where the sample rack 2031 whose measurement is completed is collected by using the conveyance area 2032. In the conveyance area 2032, the plurality of sample racks 2031 aligned in the longitudinal direction is moved in the direction D1 by the drive mechanism 4.

In order to move the sample container 2035 held by the sample rack 2031 to a predetermined sampling position, the rack sampler 203 is provided with a draw-in area 2033 for drawing the sample rack 2031 from the conveyance area 2032. The sampling position is provided, for example, at a position where the moving track of the sampling probe 207 in the vertical direction and the movement track of the opening of the sample container 2035 supported by the rack sampler 203 and held by the sample rack 2031 intersect. In the draw-in area 2033, the conveyed sample rack 2031 is moved in the direction D2 by the drive mechanism 4.

In addition, the rack sampler 203 is provided with a return area 2034 for returning the sample rack 2031 holding the sample container 2035 in which the sample is sucked to the conveyance area. In the return area 2034, the sample rack 2031 is moved in the direction D3 by the drive mechanism 4.

The reagent storage 204 holds a plurality of reagent containers 100 containing a standard liquid, a reagent used in each test item performed on a blood specimen, and the like while refrigerating. A rotary table is rotatably provided in the reagent storage 204. The rotary table places and holds the plurality of reagent containers 100 in an annular shape. In the present embodiment, although not illustrated in FIG. 2, the reagent storage 204 is covered with a detachable reagent cover.

The sampling arm 206 is provided between the reaction disk 201 and the rack sampler 203. The sampling arm 206 can be vertically moved and horizontally rotated by the drive mechanism 4. The sampling arm 206 holds the sampling probe 207 at one end.

The sampling probe 207 rotates along an arc-shaped rotational trajectory as the sampling arm 206 rotates. A sampling position for sucking a sample from the sample container 2035 held by the sample rack 2031 on the rack sampler 203 is provided on the rotational trajectory. In addition, a sample dispensing position for dispensing the sample sucked by the sampling probe 207 into the reaction container 2011 is seton the rotational trajectory of the sampling probe 207. The sample dispensing position corresponds to, for example, an intersection of a rotational trajectory of the sampling probe 207 and a movement track of the reaction container 2011 held on the reaction disk 201.

The sampling probe 207 is driven by the drive mechanism 4 and moves in the vertical direction at the sampling position or the sample dispensing position. The sampling probe 207 sucks a sample from the sample container 2035 located immediately below the sampling position according to control of a suction control function to be described later. In accordance with the control of the suction control function, the sampling probe 207 dispenses the sucked sample into the reaction container 2011 located immediately below the sample dispensing position.

The reagent dispensing arm 208 is provided between the reaction disk 201 and the reagent storage 204. The reagent dispensing arm 208 can be vertically moved and horizontally rotated by the drive mechanism 4. The reagent dispensing arm 208 holds the reagent dispensing probe 209 at one end.

The reagent dispensing probe 209 rotates along an arc-shaped rotational trajectory as the reagent dispensing arm 208 rotates. A reagent suction position is provided on the rotational trajectory. The reagent suction position is provided, for example, at a position where a rotational trajectory of the reagent dispensing probe 209 and a movement track of an opening of the reagent container 100 annularly placed on the rotary table of the reagent storage 204 intersect. In addition, a reagent dispensing position for dispensing the reagent sucked by the reagent dispensing probe 209 into the reaction container 2011 is set on the rotational trajectory of the reagent dispensing probe 209. The reagent dispensing position corresponds to, for example, an intersection of a rotational trajectory of the reagent dispensing probe 209 and a movement track of the reaction container 2011 held on the reaction disk 201.

The reagent dispensing probe 209 is driven by the drive mechanism 4 and moves in the vertical direction at the reagent suction position and the reagent dispensing position on the rotational trajectory. In addition, the reagent dispensing probe 209 sucks a reagent from the reagent container 100 stopped at the reagent suction position under the control of the control circuitry 9. In accordance with the control of the control circuitry 9, the reagent dispensing probe 209 dispenses the sucked reagent into the reaction container 2011 located immediately below the reagent dispensing position.

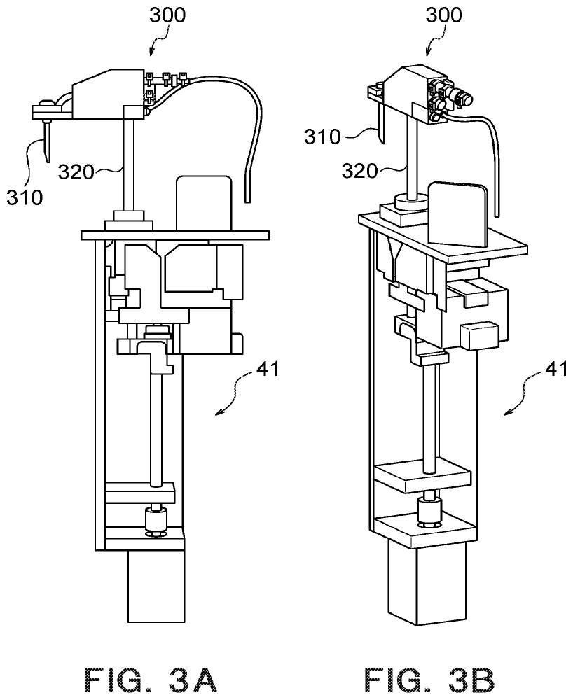

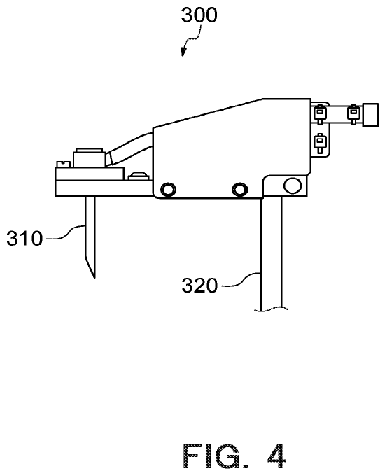

Further, the analysis mechanism 2 according to the present embodiment illustrated in FIG. 2 includes a piercing arm 300, a piercer needle 310, and a piercer drive shaft 320. The configurations of the piercing arm 300, the piercer needle 310, and the piercer drive shaft 320 will be described in detail with reference to FIGS. 2 to 4. FIGS. 3A and 3B are perspective views illustrating an example of a configuration of the piercing arm 300 and the piercing drive mechanism 41 included in the analysis mechanism 2 illustrated in FIG. 2. FIG. 4 is a diagram illustrating an example of a configuration of the piercer needle 310 included in the piercing arm 300 illustrated in FIGS. 3A and 3B.

As illustrated in FIG. 2, the piercing arm 300 is, similarly to the sampling arm 206, provided between the reaction disk 201 and the rack sampler 203. The piercing arm 300 is vertically moved and horizontally rotated by the piercing drive mechanism 41 illustrated in FIGS. 3A and 3B via the piercer drive shaft 320. As illustrated in FIGS. 3A to 4, the piercing arm 300 holds the piercer needle 310 at one end, and the other end is attached to the piercer drive shaft 320.

As illustrated in FIG. 2, the piercer needle 310 rotates along an arc-shaped rotational trajectory along with the rotation of the piercing arm 300. The piercer needle 310 pierces a cap that seals the upper surface of the sample container 2035 for sucking the sample of the sampling probe 207. In the present embodiment, the cap is configured by, for example, a rubber plug so that the piercer needle 310 can pierce the cap. A sample suction position common to the sampling arm 206 is located on the rotational trajectory of the piercing arm 300. The piercer needle 310 is driven by the drive mechanism 4 and moves in the vertical direction at the sample suction position to pierce the cap.

The piercer drive shaft 320 is attached to the piercing drive mechanism 41. Further, as illustrated in FIGS. 2 to 4, the piercing arm 300 is attached to the piercer drive shaft 320. The piercer drive shaft 320 moves the piercing arm 300 up and down in the vertical direction or rotates the piercing arm 300 in the horizontal direction according to the driving of the piercing drive mechanism 41.

Furthermore, in the analysis mechanism 2 according to the present embodiment, the same number of photometric units as the reaction containers 2011 that can be held on the reaction disk 201 are provided in the analysis mechanism 2. In the present embodiment, the photometric unit irradiates the reaction container 2011 with light, detects light transmitted through a mixed liquid of a sample and a reagent in the reaction container 2011, and detects light scattered by the mixed liquid. The photometric unit outputs the intensity of the detected light to the analysis circuitry 3 as a measurement result.

As illustrated in FIG. 1 again, the analysis circuitry 3 executes the analysis program stored in the memory 8 to implement a function corresponding to the program. For example, the analysis circuitry 3 has an analysis function 31 and a composite analysis function 32 by executing the analysis program. In the present embodiment, a case where the analysis function 31 and the composite analysis function 32 are implemented by a single processor will be described, but the present invention is not limited thereto. For example, an analysis circuitry may be configured by combining a plurality of independent processors, and the analysis function 31 and the composite analysis function 32 may be implemented by each processor executing the analysis program.

The analysis function 31 is a function of analyzing the standard data and the test data generated by the analysis mechanism 2, and is an example of an analysis unit. Specifically, for example, in the analysis function 31, the analysis circuitry 3 calculates the coagulation time based on the standard data, and generates calibration data from the calculated coagulation time. The analysis circuitry 3 outputs the generated calibration data to the control circuitry 9.

In addition, in the analysis function 31, the analysis circuitry 3 measures the coagulation process in the mixed liquid, for example, by analyzing the test data. The analysis circuitry 3 acquires a change in received light intensity for the blood coagulation reaction based on the test data. Hereinafter, the change in received light intensity is referred to as a reaction curve. The analysis circuitry 3 detects an inflection point, a saturation arrival point, and the like in the reaction curve as a coagulation end point. The inflection point, the saturation arrival point, and the like at this time are detected using a mathematical algorithm, for example, a first derivative, a second derivative, or another operation method of the reaction curve. The analysis circuitry 3 calculates a coagulation point and a coagulation time which is a time for reaching the coagulation point based on the detected coagulation end point.

In addition, depending on the test item, the analysis circuitry 3 calculates a concentration value or the like based on the calculated coagulation time and the calibration data of the test item corresponding to the test data. The analysis circuitry 3 outputs analysis data including a coagulation end point, a coagulation point, a coagulation time, a concentration value, and the like to the control circuitry 9.

The composite analysis function 32 is a function of compositing and analyzing two types of test data generated by the analysis mechanism 2, and is an example of a composite analysis unit. Specifically, in the composite analysis function 32, the analysis circuitry 3 acquires test data obtained by detecting transmitted light and test data obtained by detecting scattered light. The analysis circuitry 3 calculates information related to coagulation of the blood specimen, for example, a coagulation end point, a coagulation point, coagulation time, and the like, from the reaction curve based on the test data for transmitted light and the reaction curve based on the test data for scattered light.

The composite analysis function 32 is performed, for example, according to control from the control circuitry 9 and an analysis result in the analysis function 31. For example, the analysis circuitry 3 performs the composite analysis function 32 in response to an instruction from the control circuitry 9. In addition, for example, in the analysis function 31, in a case where the reaction is slower than expected after adding a reagent having a weak reaction, the analysis circuitry 3 performs the composite analysis function 32.

Then, the analysis circuitry 3 outputs analysis data including a coagulation end point, a coagulation point, a coagulation time, and the like to the control circuitry 9.

The control circuitry 9 illustrated in FIG. 1 executes a control program stored in the memory 8 to implement a function corresponding to the program. For example, the control circuitry 9 has a system control function 91, an imaging control function 92, a liquid level acquisition function 93, a piercing control function 94, and a suction control function 95 by executing a control program. In the present embodiment, a case where the system control function 91, the imaging control function 92, the liquid level acquisition function 93, the piercing control function 94, and the suction control function 95 are implemented by a single processor will be described, but the present invention is not limited thereto. For example, a control circuitry may be configured by combining a plurality of independent processors, and each processor may execute a control program to implement these various functions.

The system control function 91 is a function of integrally controlling each unit in the automatic analyzing apparatus 1 based on input information input from the input interface 5. For example, in the system control function 91, the control circuitry 9 controls the analysis circuitry 3 to perform analysis according to the test item.

Although described in detail later, the imaging control function 92 is a function of controlling imaging of the sample container 2035 containing the sample, the liquid level acquisition function 93 is a function of acquiring the liquid level of the sample based on the imaged image data of the sample container 2035, the piercing control function 94 is a function of controlling the piercing operation of the cap of the sample container 2035 by the piercer needle 310 by controlling the descent operation of the piercing arm 300, and the suction control function 95 is a function of controlling the suction operation by the sampling probe 207.

Note that the system control function 91, the imaging control function 92, the liquid level acquisition function 93, the piercing control function 94, and the suction control function 95 illustrated in FIG. 1 constitute a system control unit, an imaging control unit, a liquid level acquisition unit, a piercing control unit, and a suction control unit, respectively, in the present embodiment.

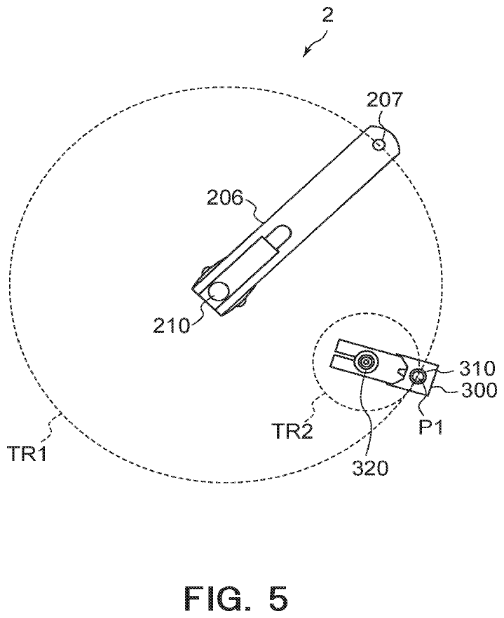

The entire configuration of the automatic analyzing apparatus 1 according to the present embodiment has been described above. Next, movement trajectories of the sampling probe 207 and the piercer needle 310 will be described with reference to FIG. 5.

FIG. 5 is a schematic view illustrating movement trajectories of the sampling probe 207 and the piercer needle 310 in the automatic analyzing apparatus illustrated in FIG. 1. An in-plane direction of the paper surface is a horizontal direction, and a direction perpendicular to the paper surface is a vertical direction. FIG. 5 schematically illustrates the operations of the sampling arm 206 and the sampling probe 207, and the piercing arm 300 and the piercer needle 310.

The sampling probe 207 is attached to a distal end portion of the sampling arm 206 so that a distal end for sucking and discharging a sample is positioned downward. The sampling arm 206 is rotationally driven about a sampling probe drive shaft 210 in a horizontal plane. When the sampling arm 206 is rotationally driven, the sampling probe 207 rotates along an arc-shaped rotational trajectory in the horizontal plane. Specifically, the sampling probe 207 moves so as to draw an arc-shaped trajectory in the horizontal plane, and a movable range thereof is indicated by a trajectory TR1.

The sampling arm 206 moves in the vertical direction as the sampling probe drive shaft 210 moves in the vertical direction. The sampling probe 207 moves in the vertical direction as the sampling arm 206 moves in the vertical direction. In the present embodiment, for example, the sampling probe 207 moves in the vertical direction at a position P1 which is a sampling position.

The piercing arm 300 is installed at a position different from the sampling arm 206 at a height lower than that of the sampling arm 206. A piercer needle 310 is held at the distal end portion of the piercing arm 300 with the distal end facing downward. Similarly to the sampling arm 206, the piercing arm 300 is rotationally driven in a horizontal plane about the piercer drive shaft 320. This rotational drive moves the piercer needle 310 along the arc-shaped track in the horizontal plane. Specifically, the piercer needle 310 moves so as to draw an arc-shaped trajectory in the horizontal plane, and a movable range thereof is indicated by a trajectory TR2.

The piercing arm 300 moves in the vertical direction as the piercer drive shaft 320 moves in the vertical direction. Then, the piercer needle 310 moves in the vertical direction as the piercing arm 300 moves in the vertical direction. In the present embodiment, for example, the piercer needle 310 moves in the vertical direction at the position P1 which is a sampling position.

The sampling position is set at a common position of the trajectory TR1 and the trajectory TR2. Specifically, the sampling position is set at the position P1 on the trajectory TR1 of the sampling probe 207 and on the trajectory TR2 of the piercer needle 310. At this position P1, piercing the cap of the sample container 2035 by the piercer needle 310 and sucking the sample by the sampling probe 207 are performed.

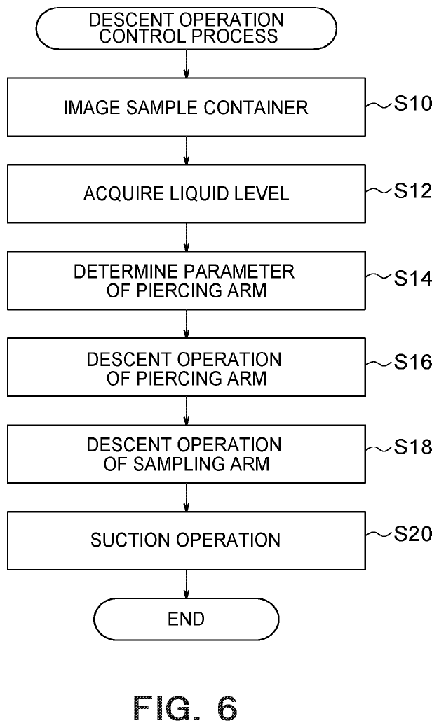

FIG. 6 is a flowchart diagram for explaining the content of a descent operation control process executed by the automatic analyzing apparatus 1 illustrated in FIG. 1. In this descent operation control process, the sample container 2035 is imaged, a parameter regarding the descent operation of the piercing arm 300 is determined based on the imaged image data of the sample container 2035, the descent operation of the piercing arm 300 is controlled based on the determined parameter, and the descent operation of the sampling arm 206 is controlled to suck the sample. The descent operation control process is a process realized by the control circuitry 9 reading and executing the descent operation control process program stored in the memory 8.

As a premise that the descent operation control process is executed, in the automatic analyzing apparatus 1 according to the present embodiment, the sample container 2035 held by the sample rack 2031 is loaded to the rack sampler 203. Loading of the sample container 2035 into the rack sampler 203 may be performed by a user or may be automatically performed by a mechanical device.

First, as illustrated in FIG. 6, in the descent operation control process executed by the automatic analyzing apparatus 1 according to the present embodiment, the automatic analyzing apparatus 1 images the sample container 2035 (Step S10). Specifically, the imaging control function 92 in the control circuitry 9 of the automatic analyzing apparatus 1 images the sample container 2035. By this imaging, the automatic analyzing apparatus 1 can acquire image data of the sample container 2035.

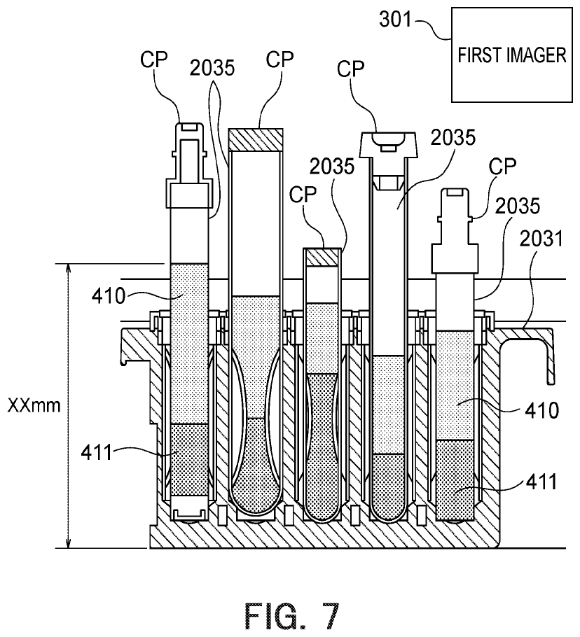

FIG. 7 is a diagram conceptually illustrating an example of a layout of the sample container 2035 on the sample rack 2031 and a first imager in the automatic analyzing apparatus 1 according to the present embodiment. As illustrated in FIG. 7, the rack sampler 203 according to the present embodiment is provided with a first imager 301. The first imager 301 can image the sample container 2035 held in the sample rack 2031, and the sample contained in the sample container 2035 is also imaged by this imaging. The imaging control function 92 images the sample container 2035 using the first imager 301 to acquire image data of the sample container 2035 and the sample contained in the sample container 2035.

The imaging of the sample container 2035 by the first imager 301 is performed once for one sample rack 2031, for example. In the example of FIG. 7, five tubes of sample containers 2035 are imaged in one imaging process. However, the imaging of the sample container 2035 by the first imager 301 may be performed once for each sample container 2035. In this case, in the example of FIG. 7, five times of imaging are performed in one sample rack 2031.

Next, as illustrated in FIG. 6, the automatic analyzing apparatus 1 according to the present embodiment acquires the liquid level height of the sample contained in the sample container 2035 based on the image data of the sample container 2035 imaged by the first imager 301 (Step S12). Specifically, the liquid level acquisition function 93 in the control circuitry 9 of the automatic analyzing apparatus 1 acquires the liquid level height of the sample contained in the sample container 2035 based on the image data of the sample container 2035. The image data of the sample container 2035 imaged by the first imager corresponds to first image data in the present embodiment.

As illustrated in FIG. 7, in the present embodiment, the automatic analyzing apparatus 1 calculates the liquid level height of the sample contained in the sample container 2035 by analyzing the image data. Here, for example, the bottom surface of the sample rack 2031 is set as a reference position, and the liquid level height of the sample is indicated as, for example, XXmm, calculating the height from the reference position.

In the present embodiment, blood that is a collected sample is separated into blood plasma and blood cells in advance using a centrifuge. That is, in FIG. 7, the upper layer portion of the sample shows blood plasma 410, and the lower layer portion of the sample shows blood cells 411. In the example illustrated in FIG. 7, the liquid level acquisition function 93 of the control circuitry 9 calculates the height from the bottom surface of the sample rack 2031 that is the reference position to the liquid level of the blood plasma 410 as XXmm. Note that the sample contained in the sample container 2035 is not limited to the blood plasma 410 and the blood cells 411, and there are various samples depending on the sample to be examined and the contents of the pretreatment. For example, blood as a sample may be separated into blood serum and blood clot in the sample container 2035, rather than the blood plasma 410 and the blood cells 411.

Note that the number of times of imaging by the first imager 301 in Step S10 described above is not limited to one time, and imaging may be performed a plurality of times. For example, a plurality of pieces of image data may be acquired by imaging the sample container 2035 at a plurality of different timings or at a plurality of different angles. In a case where a plurality of pieces of image data is acquired, in Step S12, the liquid level acquisition function 93 can analyze the plurality of pieces of image data and acquire the liquid level height with higher accuracy.

Next, as illustrated in FIG. 6, the automatic analyzing apparatus 1 according to the present embodiment determines a parameter related to the descent operation of the piercing arm 300 that holds the piercer needle 310 that pierces the cap of the sample container 2035 based on the liquid level height acquired by the liquid level acquisition function 93 (Step S14). Specifically, the piercing control function 94 in the control circuitry 9 of the automatic analyzing apparatus 1 determines the parameter related to the descent operation of the piercing arm 300. The parameter related to a descent operation of the piercing arm 300 corresponds to a first parameter in the present embodiment.

As illustrated in FIG. 7, in the present embodiment, the piercing control function 94 in the control circuitry 9 specifies the descent position of the distal end portion of the piercer needle 310 based on the liquid level height XXmm of the sample contained in the sample container 2035. By specifying this descent position, the descending amount of the piercing arm 300 can be specified.

Note that the parameter related to the descent operation of the piercing arm 300 may include not only the descending amount of the piercing arm 300 but also other elements related to the descent operation of the piercing arm 300. For example, the parameter related to the descent operation of the piercing arm 300 may include the descent speed to the descent position, the acceleration at the time of starting the descent, and the deceleration at the time of stopping the descent. That is, it can be expressed that the parameter related to the descent speed determined by the piercing control function 94 in the control circuitry 9 according to the present embodiment includes at least the descending amount of the piercing arm 300.

Next, as illustrated in FIG. 6, the automatic analyzing apparatus 1 according to the present embodiment controls the descent operation of the piercing arm 300 based on the parameter determined in Step S14 (Step S16). Specifically, the piercing control function 94 in the control circuitry 9 of the automatic analyzing apparatus 1 controls the descent operation of the piercing arm 300 based on the parameter determined in Step S14 to lower the piercer needle 310.

Next, as illustrated in FIG. 6, the automatic analyzing apparatus 1 according to the present embodiment controls the descent operation of the sampling arm 206 (Step S18). Specifically, the suction control function 95 in the control circuitry 9 of the automatic analyzing apparatus 1 controls the descent operation of the sampling arm 206 to lower the sampling probe 207. In the present embodiment, the suction control function 95 in the control circuitry 9 controls the sampling arm 206 based on the liquid level detection using the capacitance, and lowers the sampling probe 207 by a certain amount from the position where the liquid level of the sample is detected.

Next, as illustrated in FIG. 6, the automatic analyzing apparatus 1 according to the present embodiment sucks the sample (Step S20). Specifically, the suction control function 95 in the control circuitry 9 of the automatic analyzing apparatus 1 sucks a predetermined amount of sample from the distal end portion of the sampling probe 207 held by the sampling arm 206.

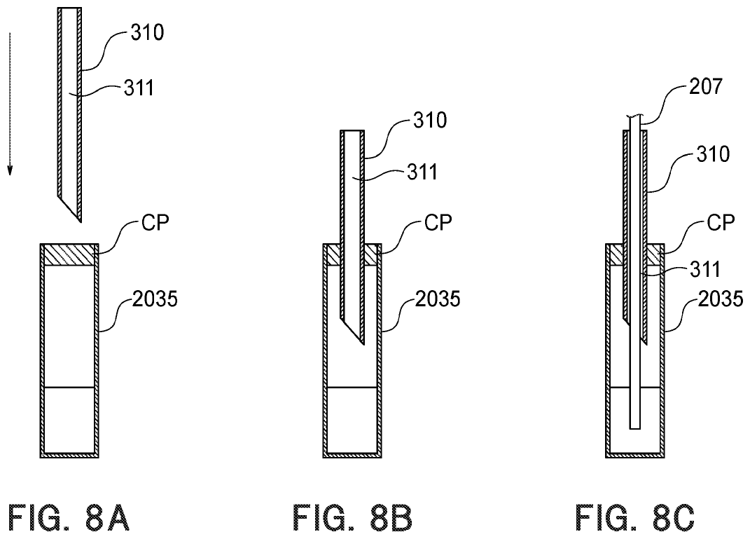

FIGS. 8A to 8C are views for explaining how the piercer needle 310 and the sampling probe 207 descend in order to perform a piercing operation by the piercer needle 310 and a suction operation by the sampling probe 207. As illustrated in FIG. 8A, when the parameter related to the descent operation of the piercing arm 300 is determined in Step S14, the piercing control function 94 lowers the piercer needle 310 by lowering the piercing arm 300 in Step S16. As a result, as illustrated in FIG. 8B, the piercer needle 310 pierces a cap CP of the sample container 2035 and descends to the descent position of the distal end portion of the piercer needle 310. That is, as illustrated in FIG. 8B, the piercer needle 310 is positioned above the liquid level of the sample contained in the sample container 2035 while piercing the cap CP of the sample container 2035. Then, as illustrated in FIG. 8C, the suction control function 95 lowers the sampling probe 207 so that the sampling probe 207 passes through a communication hole 311 of the piercer needle 310 by the descent operation of the sampling arm 206 in Step S18, immerses the distal end portion of the sampling probe 207 in the sample contained in the sample container 2035, and sucks the sample contained in the sample container 2035 in Step S20.

By the suction operation in Step S20, the descent operation control process according to the present embodiment ends. Thereafter, when the suction of the sample is completed, the suction control function 95 raises the sampling probe 207 and pulls up the sampling probe 207 from the communication hole 311 of the piercer needle 310. Then, the piercer needle 310 is pulled out from the cap CP, and a series of operations for suction of the sample is terminated. Then, the suction control function 95 discharges the sucked sample to the reaction container 2011 and analyzes the sample by the above-described analysis operation. In addition, after the cleaning of the sampling probe 207, a descent operation control process of piercing the cap CP of the next sample container 2035 and sucking the sample in the next sample container 2035 is executed.

As described above, in the automatic analyzing apparatus 1 according to the present embodiment, the first imager 301 images the sample container 2035 containing the sample, and determines the parameter related to the descent operation of the piercing arm 300 based on the image data obtained by the imaging. Therefore, the piercer needle 310 can be lowered to an appropriate position. Therefore, even in a case where the sample container 2035 and the cap CP in which the descending amount of the piercing arm 300 is not set are used, a user can use the sample container 2035 and the cap CP in which the descending amount of the piercing arm 300 is not set without setting the descending amount of the piercing arm 300, so that the work load of the user can be reduced. In addition, since the time for setting the descending amount of the piercing arm 300 can be reduced, the overall throughput of the automatic analyzing apparatus 1 can be improved.

### Second Embodiment

Various forms can be considered for the rack sampler 203 included in the automatic analyzing apparatus 1 according to the first embodiment described above. In any form of the rack sampler 203, it is sufficient that the first imager 301 images the sample container 2035 and generates image data after the sample container 2035 gets under the control of the automatic analyzing apparatus 1 and before the piercing arm 300 starts the descent operation.

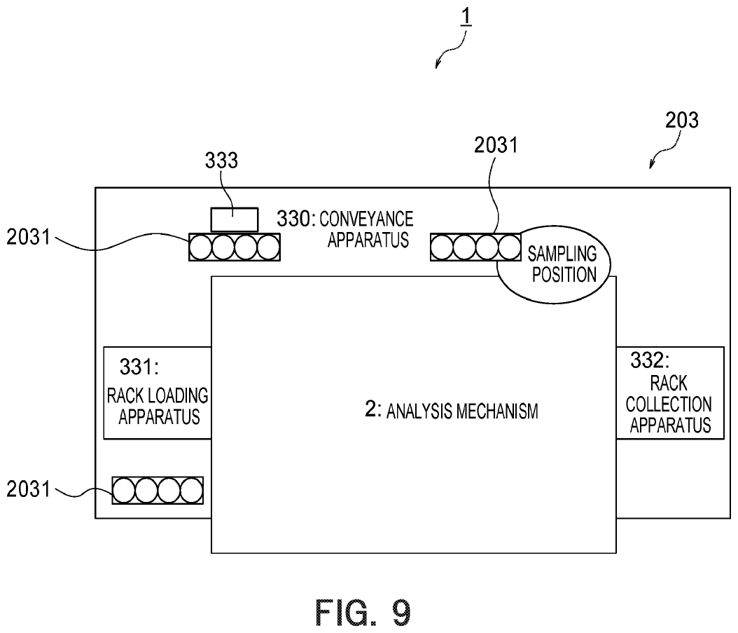

FIG. 9 is a diagram schematically illustrating one of the forms of the rack sampler 203 included in the automatic analyzing apparatus 1 according to the first embodiment described above as an automatic analyzing apparatus 1 according to the second embodiment. As illustrated in FIG. 9, the rack sampler 203 according to the present embodiment includes a conveyance apparatus 330 that conveys the sample rack 2031, a rack loading apparatus 331 that inputs the sample rack 2031 to the conveyance apparatus 330, and a rack collection apparatus 332 that collects the sample rack 2031 from the conveyance apparatus 330.

The user loads the sample rack 2031 into the conveyance apparatus 330 from the rack loading apparatus 331. The sample rack 2031 may be loaded mechanically by the rack loading apparatus 331, or may be loaded by the user using the rack loading apparatus 331 as work. On the other hand, the user collects the sample rack 2031 from the conveyance apparatus 330 by the rack collection apparatus 332. The sample rack 2031 may be collected mechanically by the rack collection apparatus 332, or may be collected by the user using the rack collection apparatus 332 as work.

In the conveyance apparatus 330, a robot arm 333 grips the sample rack 2031 and transports the sample rack 2031 and the sample container 2035 held by the sample rack 2031 to a sampling position where piercing the cap CP of the sample container 2035 by the piercer needle 310 and sucking the sample by the sampling probe 207 are performed. The number of robot arms 333 is arbitrary, and one robot arm 333 may convey the sample container 2035 loaded by the rack loading apparatus 331 to the sampling position, and may further convey the sample container to the rack collection apparatus 332 after the sampling is completed. Alternatively, the plurality of robot arms 333 may operate in cooperation to similarly convey the sample rack 2031.

In the rack sampler 203 having such a configuration, for example, the first imager 301 can image the sample container 2035 when the sample rack 2031 is loaded by the rack loading apparatus 331. In addition, the first imager 301 may image the sample container 2035 at a sampling position where the piercing of the cap CP of the sample container 2035 by the piercer needle 310 and the suction of the sample by the sampling probe 207 are performed, or may image the sample container 2035 before the sampling position.

In addition, an image reading section may be set while the sample container 2035 is being conveyed by the robot arm 333 from the rack loading apparatus 331 to the sampling position, and the first imager 301 may image the sample container 2035 in the image reading section. As described above, the position and timing at which the first imager 301 images the sample container 2035 can be arbitrarily set after the sample container 2035 gets under the control of the automatic analyzing apparatus 1 and before the piercing arm 300 starts the descent operation.

### Third Embodiment

In the automatic analyzing apparatus 1 according to each of the above-described embodiments, when the piercer needle 310 pierces the cap CP of the sample container 2035, the cap CP of the sample container 2035, the distal end portion of the piercer needle 310, and the sample contained in the sample container 2035 around the distal end portion can be imaged. In the automatic analyzing apparatus 1 according to the third embodiment, it is also possible to determine whether or not the piercer needle 310 normally penetrates the cap CP of the sample container 2035 based on the imaged image data, and in a case where the piercer needle 310 does not normally penetrate the cap CP of the sample container 2035, it is also possible to warn the user of the fact. In addition, it is also possible to store image data imaged by a second imager to be described later or transmit the image data externally for retroactive analysis. Hereinafter, the third embodiment will be described by exemplifying a case where the present modification is applied to the first embodiment described above, but the present modification can be similarly applied to other embodiments.

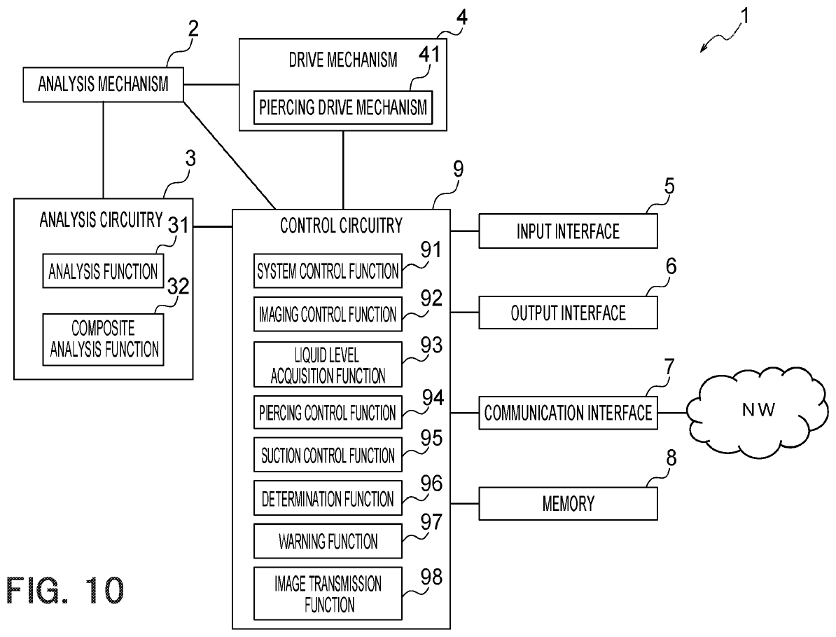

FIG. 10 is a block diagram illustrating an example of a functional configuration of an automatic analyzing apparatus according to the third embodiment, and is a diagram corresponding to FIG. 1 according to the first embodiment described above. As illustrated in FIG. 10, in the automatic analyzing apparatus 1 according to the present embodiment, the control circuitry 9 additionally includes a determination function 96, a warning function 97, and an image transmission function 98. Similarly to the other functions, the determination function 96, the warning function 97, and the image transmission function 98 are functions realized by the control circuitry 9 reading and executing a program stored in the memory 8.

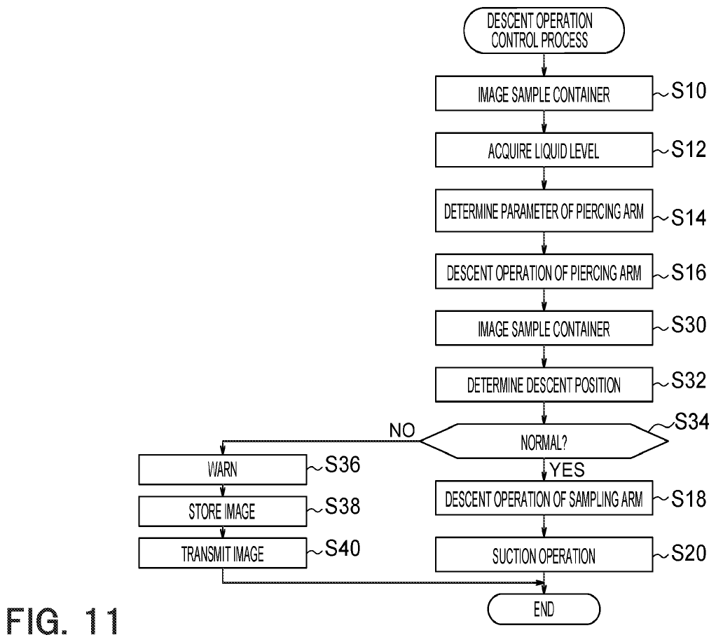

FIG. 11 is a diagram indicating a flowchart for explaining the content of a descent operation control process executed by the automatic analyzing apparatus 1 illustrated in FIG. 10, and is a diagram corresponding to FIG. 6 in the first embodiment described above. As illustrated in FIG. 11, the descent operation control process according to the present embodiment is similar to the process in the first embodiment described above until the descent operation of the piercing arm 300 in Step S16.

After Step S16, the automatic analyzing apparatus 1 images the sample container 2035 (Step S30). Specifically, the imaging control function 92 in the control circuitry 9 of the automatic analyzing apparatus 1 images the cap CP of the sample container 2035, the position where the distal end portion of the piercer needle 310 reaches when the piercer needle 310 pierce the cap CP of the sample container 2035 and the sample around the distal end portion.

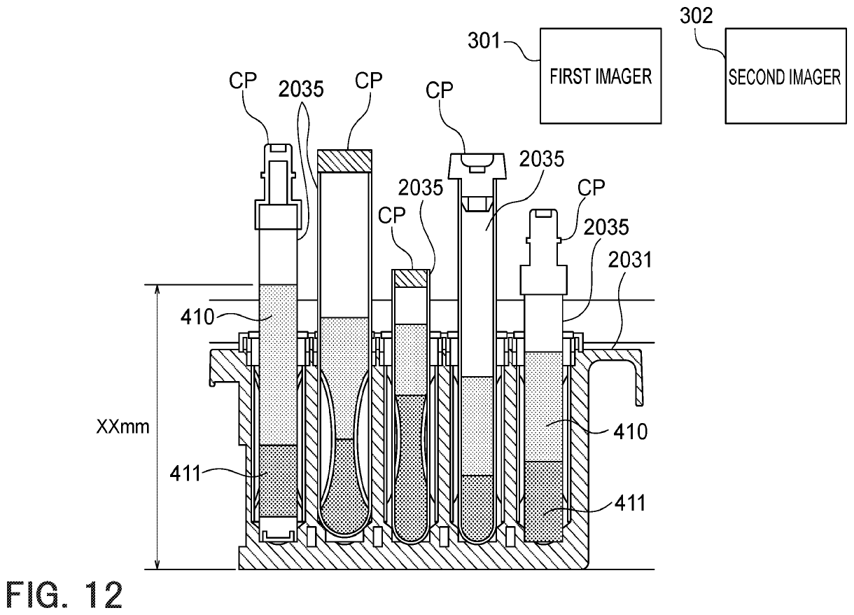

FIG. 12 is a diagram conceptually illustrating an example of a layout of the sample container 2035, the first imager 301, and a second imager on the sample rack 2031 in the automatic analyzing apparatus 1 according to the third embodiment. As illustrated in FIG. 12, a second imager 302 images the sample container 2035 after the piercer needle 310 descended. The position where the second imager 302 is provided is arbitrary as long as the descent position of the piercer needle 310 can be specified, but in order to specify the descent position of the piercer needle 310, it is necessary to provide the second imager 302 in the vicinity of the sampling position where the piercing is performed by the piercer needle 310.

Furthermore, the second imager 302 is not necessarily provided separately from the first imager 301. That is, the automatic analyzing apparatus 1 can be configured so that the first imager 301 also serves as the second imager 302.

Next, as illustrated in FIG. 11, it is determined whether or not the piercer needle 310 normally penetrates the cap CP of the sample container 2035 based on the image data imaged by the second imager 302 (Step S32). Specifically, the determination function 96 in the control circuitry 9 of the automatic analyzing apparatus 1 performs this determination process. The determination function 96 in Step S32 constitutes a first determination unit in the present embodiment. Here, the fact that the piercer needle 310 normally penetrates the cap CP of the sample container 2035 means that the piercer needle 310 penetrates the cap CP of the sample container 2035, and the distal end portion of the piercer needle 310 is positioned above the liquid level of the sample contained in the sample container 2035. The image data imaged by the second imager corresponds to second image data in the present embodiment.

That is, the determination function 96 in the control circuitry 9 performs image analysis on the image data acquired in Step S30, so that it is possible to specify the position where the distal end portion of the piercer needle 310 is lowered in Step S18. In addition, the periphery of the distal end portion of the piercer needle 310 can also be specified. Therefore, the determination function 96 in the control circuitry 9 can determine whether or not the piercer needle 310 is lowered and normally penetrates the cap CP of the sample container 2035.

Next, as illustrated in FIG. 11, as a result of the determination in Step S32, the automatic analyzing apparatus 1 determines whether or not the piercer needle 310 normally penetrates the cap CP of the sample container 2035 (Step S34). Specifically, the determination function 96 in the control circuitry 9 of the automatic analyzing apparatus 1 performs this determination process.

Then, when it is determined in Step S34 that the piercer needle 310 normally penetrates the cap CP of the sample container 2035 (Step S34: Yes), the process of controlling the descent operation of the sampling arm 206 (Step S18) is executed as in the first embodiment. The subsequent descent operation control process is similar to that of the first embodiment described above.

On the other hand, when it is determined that the piercer needle 310 does not normally penetrate the cap CP of the sample container 2035 (Step S34: No), the automatic analyzing apparatus 1 warns that (Step S36). Specifically, the warning function 97 in the control circuitry 9 of the automatic analyzing apparatus 1 outputs a warning that the piercer needle 310 does not normally penetrate the cap CP of the sample container 2035.

For example, the warning in Step S36 may be displayed by a display circuitry provided as the output interface 6, or may be printed by a print circuitry. The warning function 97 that executes Step S36 constitutes a warning unit in the present embodiment.

Next, as illustrated in FIG. 11, the automatic analyzing apparatus 1 stores the image data imaged by the second imager 302 (Step S38). For example, the automatic analyzing apparatus 1 stores the image data imaged by the second imager 302 in the memory 8. The user can investigate the cause of the piercer needle 310 not normally penetrating the cap CP of the sample container 2035 by retroactively analyzing the image data stored in the memory 8. The memory 8 in which the image data is stored constitutes an image memory in the present embodiment.

The image data stored in the memory 8 is held until deleted at an arbitrary timing by the user. Alternatively, the image data stored in the memory 8 can be automatically deleted, for example, after a predetermined period has elapsed. The period during which the image data is stored in the memory 8 may be fixedly set in advance, or may be arbitrarily set by the user.

Next, as illustrated in FIG. 11, the automatic analyzing apparatus 1 transmits the image data imaged by the second imager 302 to the outside of the automatic analyzing apparatus 1 (Step S40). Specifically, the image transmission function 98 in the control circuitry 9 of the automatic analyzing apparatus 1 transmits the image data to the outside of the automatic analyzing apparatus 1 via the communication interface 7. The image transmission function 98 constitutes an image transmission unit in the present embodiment.

For example, the image transmission function 98 in the control circuitry 9 may transmit the image data imaged by the second imager 302 to an online maintenance computer provided by a manufacturer of the automatic analyzing apparatus 1, or to a computer of a service department of the automatic analyzing apparatus 1.

Then, the descent operation control process according to the present embodiment ends. That is, when the piercer needle 310 does not normally penetrate the cap CP of the sample container 2035, the sample is not analyzed, and the descent operation control process for the next sample is performed.

As described above, according to the automatic analyzing apparatus 1 of the present embodiment, since the determination function 96 in the control circuitry 9 is additionally provided, the automatic analyzing apparatus 1 can determine whether or not the piercer needle 310 normally penetrates the cap CP of the sample container 2035, based on the image data imaged by the second imager 302.

In addition, according to the automatic analyzing apparatus 1 according to the present embodiment, in a case where the piercer needle 310 does not normally penetrate the cap CP of the sample container 2035, a warning is issued to the user, so that the user can quickly grasp the occurrence of abnormality. Furthermore, according to the automatic analyzing apparatus 1 of the present embodiment, it is possible to store image data imaged by the second imager 302 or transmit the image data externally for retroactive analysis, and it is possible to expect a quick and accurate response in the case of the occurrence of abnormality.

Note that it has been assumed that the automatic analyzing apparatus 1 according to the present embodiment includes both the image memory that stores the image data and the image transmission unit that transmits the image data to the outside, but the automatic analyzing apparatus 1 may include one of the image memory and the image transmission unit. In other words, the automatic analyzing apparatus 1 can be configured to include at least one of the image memory and the image transmission unit.

### Fourth Embodiment

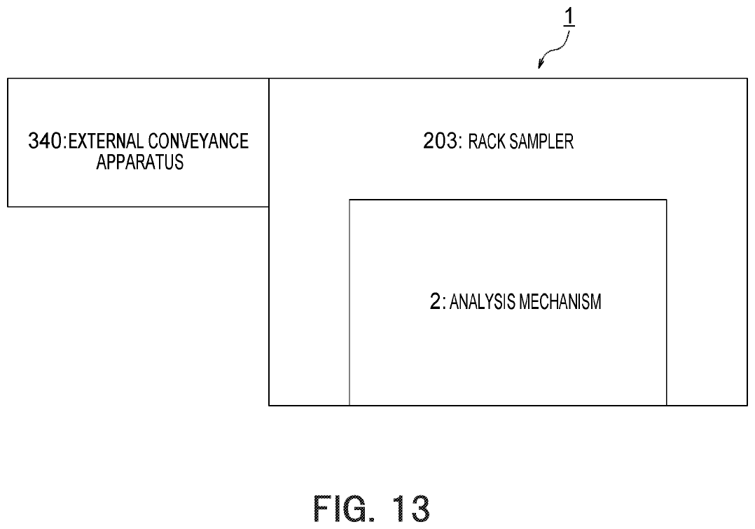

The automatic analyzing apparatus 1 according to each embodiment described above can also be connected to an external conveyance apparatus provided to the outside of the automatic analyzing apparatus 1. FIG. 13 is a diagram illustrating an example of a configuration in a case where an external conveyance apparatus is connected to an automatic analyzing apparatus 1 according to the fourth embodiment. In other words, FIG. 13 illustrates an automatic analysis system including the automatic analyzing apparatus 1 and an external conveyance apparatus 340 connected to the automatic analyzing apparatus 1.

As illustrated in FIG. 13, the rack sampler 203 of the automatic analyzing apparatus 1 is connected to the external conveyance apparatus 340. The external conveyance apparatus 340 transports the sample container 2035 to the rack sampler 203 of the automatic analyzing apparatus 1. The sample container 2035 conveyed to the rack sampler 203 is conveyed to the sampling position where the piercing by the piercer needle 310 and the sampling of the sample by the sampling probe 207 are performed, and the descent operation control process is executed in the automatic analyzing apparatus 1 as in each of the above-described embodiments.

In the automatic analysis system having such a configuration, the first imager 301 can be provided in the external conveyance apparatus 340. Then, the first imager 301 images the sample container 2035 while the sample container 2035 is being transported by the external conveyance apparatus 340.

In the external conveyance apparatus 340, when the sample container 2035 is transported while being held by the sample rack 2031, the liquid level acquisition function 93 in the control circuitry 9 can calculate the liquid level height of the sample with the bottom surface of the sample rack 2031 as a reference position, as in each embodiment described above.

On the other hand, in the external conveyance apparatus 340, when the sample container 2035 is conveyed without being held by the sample rack 2031, the liquid level acquisition function 93 in the control circuitry 9 may calculate the liquid level height of the sample from the conveyance surface with the conveyance surface of the external conveyance apparatus 340 as a reference position.

As described above, even in a case where the external conveyance apparatus 340 is additionally connected to the automatic analyzing apparatus 1 according to each of the above-described embodiments, the automatic analyzing apparatus 1 executes the descent operation control process of FIG. 6 described above, whereby the normal descent operation of the piercing arm 300 can be realized even in a case where the sample container 2035 and the cap CP for which the descending amount of the piercing arm 300 is not set are conveyed.

### Fifth Embodiment

In the automatic analyzing apparatus 1 according to each embodiment described above, the sample container 2035 is held by the sample rack 2031 and stored in the rack sampler 203. However, the sample container 2035 may be configured to be stored in a disk sampler instead of the rack sampler 203. A configuration of an automatic analyzing apparatus 1 including such a disk sampler will be described as a fifth embodiment.

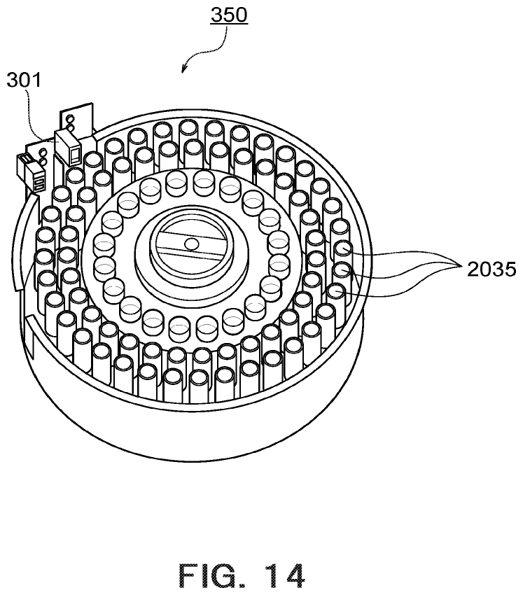

FIG. 14 is a diagram illustrating an example of a configuration of a disk sampler that stores the sample container 2035 in the automatic analyzing apparatus 1 according to the fifth embodiment. The disk sampler 350 illustrated in FIG. 14 is provided inside the automatic analyzing apparatus 1, and is configured to be able to arrange the sample container 2035 circumferentially on a disc-shaped disk. For example, the user sets the sample container 2035 in the automatic analyzing apparatus 1 by putting the sample container 2035 in the disk sampler 350.

In the example of FIG. 14, the first imager 301 is provided at the outer peripheral position of the disk sampler 350. As described above, the first imager 301 images the sample container 2035. Note that the arrangement of the first imager 301 is arbitrary, and the first imager 301 can be arranged at any position as long as the sample container 2035 and the contained sample can be imaged.

The sampling arm 206 holding the sampling probe 207 that sucks the sample repeats the above-described rotation operation and vertical operation with respect to the sample container 2035 arranged on the disk sampler 350, thereby realizing an operation of sucking the sample contained in the sample container 2035 and discharging the sample to the reaction container 2011.

As described above, in a structure where the disk sampler 350 is included as the automatic analyzing apparatus 1 according to the present embodiment, the automatic analyzing apparatus 1 executes the descent operation control process of FIG. 6 described above, whereby the normal descent operation of the piercing arm 300 can be realized even in a case where the sample container 2035 for which the descending amount of the piercing arm is not set are conveyed.

### Sixth Embodiment

In the automatic analyzing apparatus 1 according to each of the above-described embodiments, the information related to the sample rack 2031 may be acquired from the image data imaged by the first imager 301, or may be acquired base on the information of a barcode read by a barcode reader by providing the barcode reader in the rack sampler 203. An example in which the automatic analyzing apparatus 1 acquires information related to the sample rack 2031 in use by various methods will be described as a sixth embodiment.

The case where the information related to the sample rack 2031 in use is acquired from the image data imaged by the first imager 301 can be realized by performing image analysis of the image data. For example, the liquid level acquisition function 93 in the control circuitry 9 can acquire the structure and size of the sample rack 2031 by image analysis and specify the type of the sample rack 2031.

On the other hand, the information related to the sample rack 2031 in use can also be acquired from data other than the image data imaged by the first imager 301.

When the information related to the sample rack 2031 in use is acquired by barcode reading by the barcode reader provided in the rack sampler 203, the barcode attached to the sample rack 2031 is read, and the information related to the sample rack 2031 is acquired based on the read barcode information. For example, when the barcode attached to the sample rack 2031 includes information related to the type and size of the sample rack 2031, the liquid level acquisition function 93 in the control circuitry 9 can acquire the information by reading the barcode.

When the barcode attached to the sample rack 2031 includes unique identification information for specifying the sample rack 2031, the unique identification information and the information related to the sample rack 2031 are associated with each other and held by the automatic analyzing apparatus 1. Then, the liquid level acquisition function 93 in the control circuitry 9 acquires information related to the sample rack 2031 based on the unique identification information read by the barcode reader.

Furthermore, the information related to the sample rack 2031 does not necessarily need to be acquired using the barcode reader or the first imager 301, but may be preset and input by the user to the automatic analyzing apparatus 1. For example, in a case where there is one type of sample rack 2031, the user sets and inputs information related to this type to the automatic analyzing apparatus 1, so that it is not necessary to acquire these pieces of information after the analysis operation of the automatic analyzing apparatus 1 starts.

### Seventh Embodiment

In the automatic analyzing apparatus 1 according to each of the above-described embodiments, the descent operation of the sampling arm 206 is controlled based on the liquid level detection using the capacitance to lower the sampling probe 207. However, in the automatic analyzing apparatus 1, it is also possible to determine a parameter related to the descent operation of the sampling arm 206 holding the sampling probe 207 based on the liquid level height acquired by the liquid level acquisition function 93, and control the descent operation of the sampling arm 206 based on the determined parameter. Hereinafter, the seventh embodiment will be described by exemplifying a case where the present modification is applied to the first embodiment described above, but the present modification can be similarly applied to other embodiments.

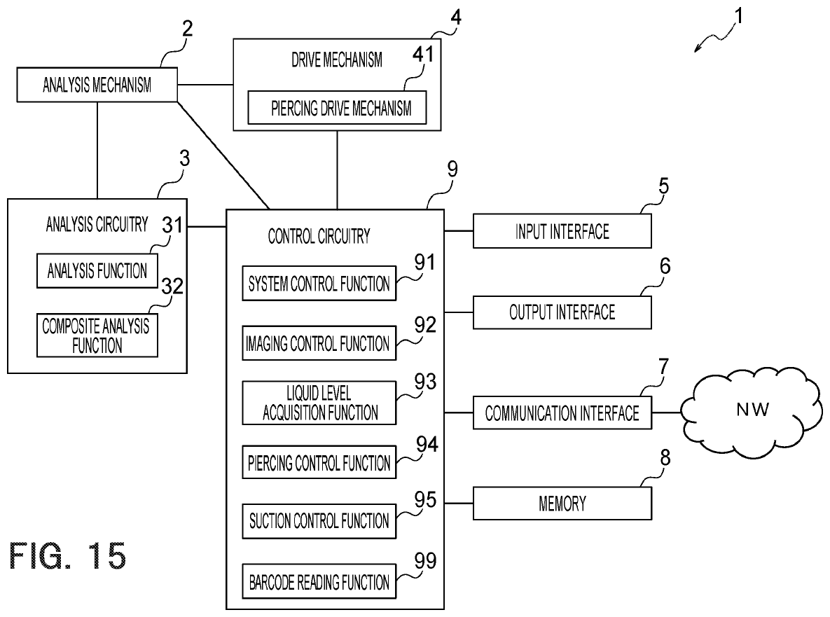

FIG. 15 is a block diagram illustrating an example of a functional configuration of an automatic analyzing apparatus according to the seventh embodiment, and is a diagram corresponding to FIG. 1 according to the first embodiment described above. As illustrated in FIG. 15, in the automatic analyzing apparatus 1 according to the present embodiment, the control circuitry 9 additionally includes a barcode reading function 99. Similarly to the other functions, the barcode reading function 99 is a function realized by the control circuitry 9 reading and executing a program stored in the memory 8.

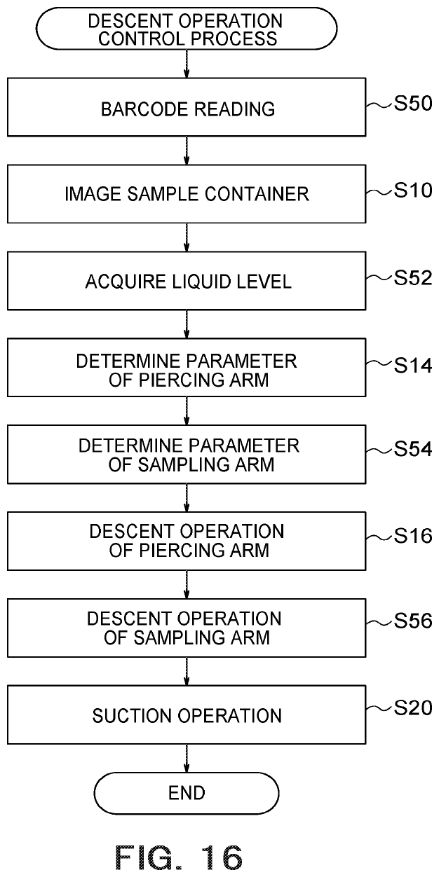

FIG. 16 is a flowchart diagram for explaining the content of a descent operation control process executed by the automatic analyzing apparatus 1 illustrated in FIG. 15, and is a diagram corresponding to FIG. 6 in the first embodiment described above. The descent operation control process is a process realized by the control circuitry 9 reading and executing the descent operation control process program stored in the memory 8.

First, as illustrated in FIG. 16, in the descent operation control process executed by the automatic analyzing apparatus 1 according to the present embodiment, the automatic analyzing apparatus 1 performs barcode reading (Step S50). Specifically, the barcode reading function 99 in the control circuitry 9 of the automatic analyzing apparatus 1 reads a barcode attached to the sample container or the sample rack 2031.

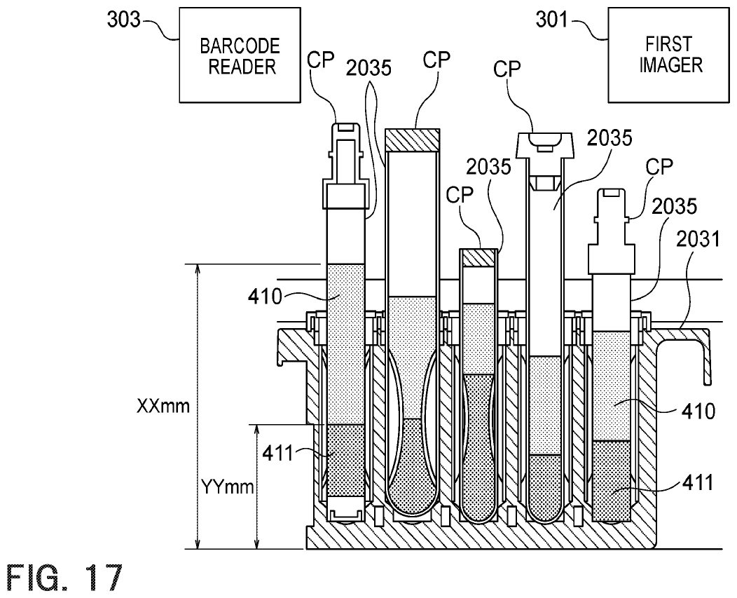

FIG. 17 is a diagram conceptually illustrating an example of a layout of the sample container 2035 on the sample rack 2031, the first imager 301, and a barcode reader in the automatic analyzing apparatus 1 according to the seventh embodiment, and is a diagram corresponding to FIG. 7 according to the first embodiment described above. As illustrated in FIG. 17, the rack sampler 203 according to the present embodiment is provided with a barcode reader 303. The barcode reader 303 can read a barcode attached to the sample container 2035 and a barcode attached to the sample rack 2031. By reading the barcode using the barcode reader 303, the barcode reading function 99 can acquire the contents of the test request of the sample container 2035 and can acquire information of the specimen as necessary. After Step S50, the imaging of the sample container 2035 in Step S10 is the same process as in the first embodiment described above.

After Step S10, the automatic analyzing apparatus 1 according to the present embodiment acquires the liquid level height of the sample contained in the sample container 2035 based on the image data of the sample container 2035 imaged by the first imager 301 (Step S52). Specifically, the liquid level acquisition function 93 in the control circuitry 9 of the automatic analyzing apparatus 1 acquires the liquid level height of the sample contained in the sample container 2035 based on the image data of the sample container 2035. The image data of the sample container 2035 imaged by the first imager corresponds to first image data in the present embodiment.

As illustrated in FIG. 17, in the present embodiment, the automatic analyzing apparatus 1 calculates the liquid level height of the sample contained in the sample container 2035 by analyzing the image data. Here, for example, the bottom surface of the sample rack 2031 is set as a reference position, and the liquid level height of the sample is indicated as, for example, XXmm, calculating the height from the reference position.

Note that in the present embodiment, the collected blood is separated into blood plasma and blood cells in advance using a centrifuge. That is, in FIG. 17, the upper layer portion of the sample shows blood plasma 410, and the lower layer portion of the sample shows blood cells 411. Therefore, the liquid level acquisition function 93 of the control circuitry 9 may acquire the type of the sample contained in the sample container 2035 and the liquid level height for each of the plurality of types of samples based on the acquired image data.

In the example of FIG. 17, the liquid level acquisition function 93 of the control circuitry 9 may calculate the height from the bottom surface of the sample rack 2031 as the reference position to the liquid level of the blood plasma 410 as XXmm, and calculate the height from the bottom surface of the sample rack 2031 as the reference position to the liquid level of the blood cells 411 as YYmm. Note that the type of sample is not limited to the blood plasma 410 and the blood cells 411, and there are various samples depending on the sample to be examined and the contents of the pretreatment. For example, as the type of sample, the sample may be separated into blood serum and blood clot in the sample container 2035, rather than the blood plasma 410 and the blood cells 411. When only one sample type is contained in the sample container, only the liquid level height of the sample may be calculated from the bottom surface of the sample rack 2031. The liquid level acquisition function 93 analyzes the image data imaged by the first imager 301 to calculate the liquid level height of each type.

Note that the number of times of imaging by the first imager 301 in Step S10 described above is not limited to one time, and imaging may be performed a plurality of times. For example, a plurality of pieces of image data may be acquired by imaging the sample container 2035 at a plurality of different timings or at a plurality of different angles. In a case where a plurality of pieces of image data is acquired, in Step S52, the liquid level acquisition function 93 can analyze the plurality of pieces of image data and acquire the liquid level height with higher accuracy. After Step S52, the process in Step S14 is similar to the descent operation control process in the first embodiment described above.

Next, as illustrated in FIG. 16, the automatic analyzing apparatus 1 according to the present embodiment determines a parameter related to the descent operation of the sampling arm 206 holding the sampling probe 207 that sucks the sample based on the liquid level height acquired by the liquid level acquisition function 93 (Step S54). Specifically, the suction control function 95 in the control circuitry 9 of the automatic analyzing apparatus 1 determines the parameter related to the descent operation of the sampling arm 206. The parameter related to a descent operation of the sampling arm 206 corresponds to a second parameter in the present embodiment.

As illustrated in FIG. 17, in the present embodiment, the suction control function 95 in the control circuitry 9 specifies the descent position of the distal end portion of the sampling probe 207 based on the liquid level height XXmm of the sample contained in the sample container 2035. By specifying this descent position, the descending amount of the sampling arm 206 can be specified. In addition, the suction control function 95 in the control circuitry 9 specifies the descent speed to the descent position. Here, the parameter of the descent speed may include not only the speed in the constant speed state but also the acceleration at the start of the descent of the sampling arm 206 and the deceleration at the stop of the descent of the sampling arm 206.

In the present embodiment, the liquid level acquisition function 93 may acquire the type of sample and the liquid level height for each type of the sample. Therefore, the suction control function 95 in the control circuitry 9 may determine the parameter related to the descent operation based on the type of the sample acquired by the liquid level acquisition function 93 and the liquid level height for each type of the sample. For example, in the example of FIG. 17, when it is necessary to suck the blood cells 411, since the liquid level height is YYmm, the suction control function 95 in the control circuitry 9 may determine the descending amount of the sampling arm 206 so that the distal end portion of the sampling probe 207 is at the position where the blood cells 411 exist.

In addition, the parameter related to the descent operation determined by the suction control function 95 in the control circuitry 9 may include not only the descending amount of the sampling arm 206 and the descent speed of the sampling arm 206 but also other elements related to the descent operation. In other words, it can be expressed that the parameter related to the descent speed determined by the suction control function 95 in the control circuitry 9 according to the present embodiment includes at least the descending amount of the sampling arm 206 and the descent speed of the sampling arm 206. After Step S54, the process in Step S16 is similar to the descent operation control process in the first embodiment described above.

Next, as illustrated in FIG. 16, the automatic analyzing apparatus 1 according to the present embodiment controls the descent operation of the sampling arm 206 based on the parameter determined in Step S54 (Step S56). Specifically, the suction control function 95 in the control circuitry 9 of the automatic analyzing apparatus 1 controls the descent operation of the sampling arm 206 based on the parameter determined in Step S54 to lower the sampling probe 207.

More specifically, also in the automatic analyzing apparatus 1 according to the present embodiment, as illustrated in FIG. 8C, the suction control function 95 causes the sampling probe 207 to descend by the descent operation of the sampling arm 206, and the distal end portion of the sampling probe 207 is immersed in the sample contained in the sample container 2035. For example, in Step S56, in the case of a test in which the blood plasma 410 needs to be sucked, the descending amount of the sampling arm 206 is set as a parameter so that the distal end portion of the sampling probe 207 reaches the blood plasma 410. Note that, in the case of a test in which the blood cells 411 need to be sucked, the descending amount of the sampling arm 206 is set as a parameter so that the distal end portion of the sampling probe 207 reaches the blood cells 411. Therefore, the distal end portion of the sampling probe 207 can descend to the position of the blood cells 411 instead of the blood plasma 410

Next, as illustrated in FIG. 16, the automatic analyzing apparatus 1 according to the present embodiment sucks the sample at a position where the descent operation of the sampling arm 206 is stopped (Step S20). Specifically, the suction control function 95 in the control circuitry 9 of the automatic analyzing apparatus 1 sucks a predetermined amount of sample from the distal end portion of the sampling probe 207 held by the sampling arm 206. By the suction operation of the sample, the descent operation control process according to the present embodiment ends.

After this descent operation control process is completed, the automatic analyzing apparatus 1 according to the present embodiment raises the sampling arm 206, discharges the sucked sample to the reaction container 2011, and analyzes the sample by the analysis operation described above. In addition, after the sampling probe 207 is cleaned, a descent operation control process for sucking the next sample is executed.

As described above, in the automatic analyzing apparatus 1 according to the present embodiment, the first imager 301 images the sample container 2035 containing the sample, and determines the parameter related to the descent operation of the sampling arm 206 based on the image data obtained by the imaging. Therefore, the sampling probe 207 can be lowered at a high speed to an appropriate position so as to suck the sample. Therefore, the number of tests that can be performed by the automatic analyzing apparatus 1 within a predetermined time can be increased, and the overall throughput of the automatic analyzing apparatus 1 can be improved.

That is, unlike the conventional case, since it is not necessary to detect the liquid level using the sampling probe 207 in the middle of descending, therefore, it is possible to realize an accurate descent operation of the sampling arm 206 capable of increasing the speed. Furthermore, since there is no time lag between the time when the sampling probe 207 detects the liquid level and the time when the sampling probe 207 stops the descent operation, even when the remaining amount of the sample is small, the sample can be sucked without the distal end portion of the sampling probe 207 colliding with the bottom of the sample container 2035.

Moreover, since the liquid level acquisition function 93 in the control circuitry 9 of the automatic analyzing apparatus 1 according to the present embodiment is configured to acquire the liquid level height for each type of sample contained in the sample container 2035, it is possible to control the descending amount of the sampling arm 206 so that a type of sample required for a test can be accurately sucked from the distal end portion of the sampling probe 207. Therefore, various types of samples contained in the sample container 2035 can be sucked according to the amount thereof.

### Eighth Embodiment

In the automatic analyzing apparatus 1 according to each of the above-described embodiments, it is determined whether or not the type of the sample container 2035 can be specified based on the image data of the sample container 2035 imaged by the first imager 301, and when the type of the sample container cannot be specified, the liquid level height of the sample contained in the sample container 2035 may be acquired based on the image data of the sample container 2035 imaged by the first imager 301. Hereinafter, the eighth embodiment will be described by exemplifying a case where the present modification is applied to the first embodiment described above, but the present modification can be similarly applied to other embodiments.

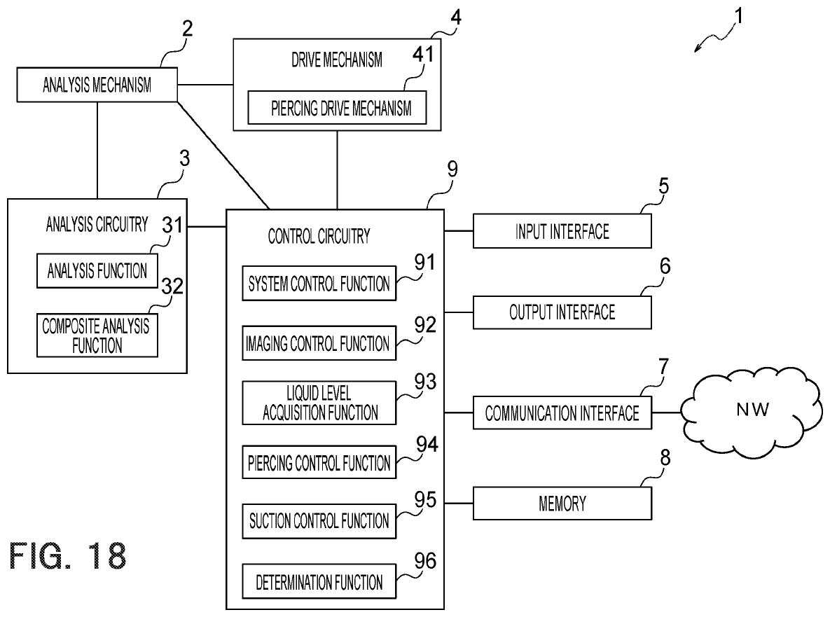

FIG. 18 is a block diagram illustrating an example of a functional configuration of an automatic analyzing apparatus 1 according to the eighth embodiment, and is a diagram corresponding to FIG. 1 according to the first embodiment described above. As illustrated in FIG. 18, in the automatic analyzing apparatus 1 according to the present embodiment, the control circuitry 9 additionally includes a determination function 96. Similarly to the other functions, the determination function 96 is a function realized by the control circuitry 9 reading and executing a program stored in the memory 8.

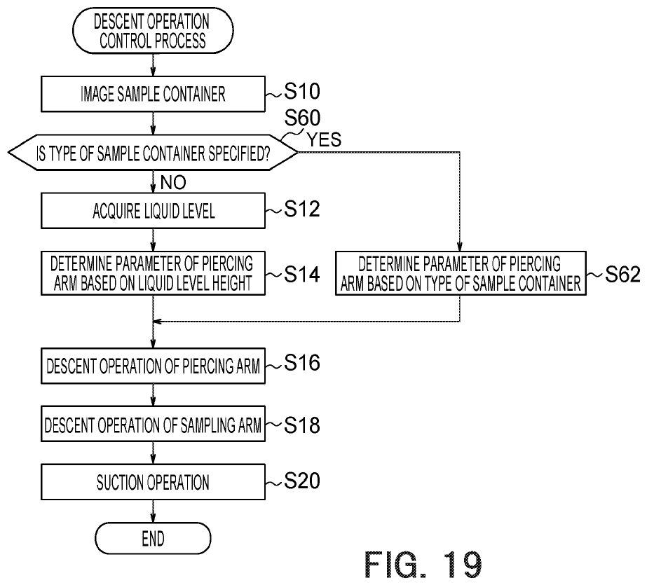

FIG. 19 is a flowchart diagram for explaining the content of a descent operation control process executed by the automatic analyzing apparatus 1 illustrated in FIG. 18, and is a diagram corresponding to FIG. 6 in the first embodiment described above. As illustrated in FIG. 19, in the descent operation control process according to the present embodiment, the process in Step S10 is similar to the process in the first embodiment described above.

After Step S10, the automatic analyzing apparatus 1 determines whether or not the type of the sample container 2035 can be specified (Step S60). Specifically, the determination function 96 in the control circuitry 9 of the automatic analyzing apparatus 1 determines whether or not the type of the sample container 2035 can be specified based on the image data imaged by the first imager 301. The determination function 96 in Step S60 constitutes a second determination unit in the present embodiment.

That is, it is determined whether or not the type of the sample container 2035 can be specified by performing image analysis on the image data of the sample container 2035 imaged in Step S10 described above, and comparing the size and shape of the sample container 2035 and/or the shape, size, color, and the like of the cap CP in the image data with the information related to the sample container 2035 or the information related to the cap CP stored in the memory 8. The image data of the sample container 2035 imaged by the first imager corresponds to first image data in the present embodiment.

Then, in Step S60, when the type of the sample container 2035 cannot be specified (Step S60: No), similarly to the first embodiment, a process of acquiring the liquid level height of the sample contained in the sample container 2035 (Step S12) is executed based on the image data of the sample container 2035 imaged by the first imager 301. The process in Steps S12 and S14 is similar to that in the first embodiment described above.

On the other hand, when the type of the sample container 2035 can be specified in Step S60 (Step S60: Yes), the automatic analyzing apparatus 1 determines a parameter related to the descent operation of the piercing arm 300 based on the type of the sample container 2035 (Step S62). Specifically, the piercing control function 94 in the control circuitry 9 of the automatic analyzing apparatus 1 acquires the parameter related to the descent operation of the piercing arm 300 based on the specified type of the sample container 2035 from the memory 8, thereby determining the parameter related to the descent operation of the piercing arm 300. The parameter related to a descent operation of the piercing arm 300 corresponds to a first parameter in the present embodiment.

The parameter related to the descent operation of the piercing arm 300 may include not only the descending amount of the piercing arm 300 but also other elements related to the descent operation. For example, the parameter related to the descent operation of the piercing arm 300 may include the descent speed to the descent position, the acceleration at the time of starting the descent, and the deceleration at the time of stopping the descent. That is, it can be expressed that the parameter related to the descent speed determined by the piercing control function 94 in the control circuitry 9 according to the present embodiment includes at least the descending amount of the piercing arm 300. In Step S62, the liquid level position may be specified. By specifying the liquid level position in this way, it is possible to be positioned above the liquid level of the sample contained in the sample container 2035 while piercing the cap CP of the sample container 2035.

Next, as illustrated in FIG. 19, the automatic analyzing apparatus 1 according to the present embodiment executes a process of controlling the descent operation of the piercing arm 300 (Step S16) based on the parameter determined in Step S14 or the parameter determined in Step S62, similarly to the first embodiment described above. The subsequent descent operation control process is similar to that of the first embodiment described above.

By the suction operation in Step S20, the descent operation control process according to the present embodiment ends. Thereafter, when the suction of the sample is completed, the suction control function 95 raises the sampling probe 207 and pulls up the sampling probe 207 from the communication hole 311 of the piercer needle 310. Then, the piercer needle 310 is pulled out from the cap CP, and a series of operations for suction of the sample is terminated. Then, the suction control function 95 discharges the sucked sample to the reaction container 2011 and analyzes the sample by the above-described analysis operation. In addition, after the cleaning of the sampling probe 207, a descent operation control process of piercing the cap CP of the next sample container 2035 and sucking the sample in the next sample container 2035 is executed.

As described above, according to the automatic analyzing apparatus 1 according to the present embodiment, the first imager 301 images the sample container 2035 containing the sample, determines whether or not the type of the sample container 2035 can be specified base on the image data obtained by the imaging, and in a case where the type of the sample container 2035 can be specified, acquires the parameter related to the descent operation of the piercing arm 300 based on the specified type of the sample container 2035 from the memory 8 to determine the parameter. Therefore, in a case where the type of the sample container 2035 can be specified, it is not necessary to acquire the liquid level height of the sample contained in the sample container 2035 based on the image data, it is possible to reduce the time required for image analysis for acquiring the liquid level height, and it is possible to improve the overall throughput of the automatic analyzing apparatus 1.

### Modification

In the automatic analyzing apparatus 1 according to each of the above-described embodiments, the first imager 301 images the sample container 2035 to acquire the liquid level height of the sample contained in the sample container 2035. Therefore, the sample container 2035 needs to have a transparent portion at least partially formed so that the liquid level of the contained sample is included in the imaged image data. That is, the liquid level height of the sample can be acquired by image analysis through the transparent portion formed in the sample container 2035.

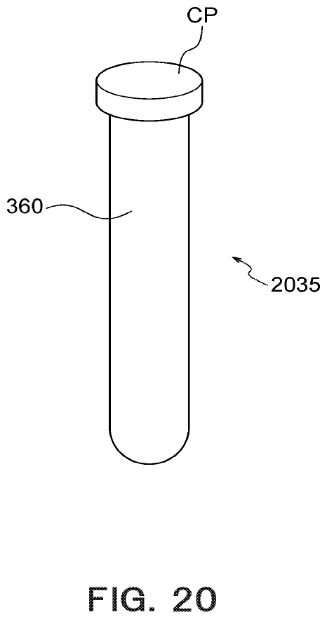

FIG. 20 is a diagram illustrating an example of a configuration of a sample container 2035 whose entire surface is formed of a transparent portion in an automatic analyzing apparatus 1 according to a modification. By configuring the entire surface of the sample container 2035 with a transparent portion 360 in this manner, the first imager 301 can image the sample container 2035 from any angle, and the liquid level height of the contained sample can be acquired based on the acquired image data. The image data of a sample container 2035 imaged by the first imager corresponds to first image data in the present modification.

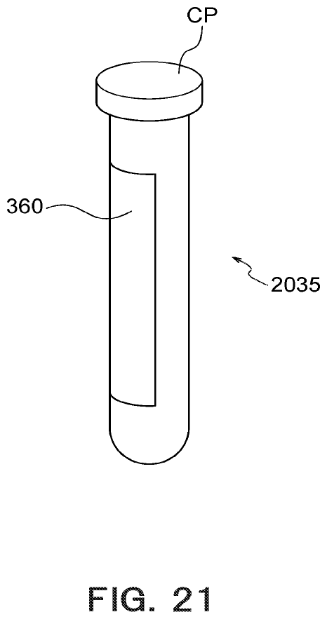

FIG. 21 is a diagram illustrating an example of a configuration of a sample container 2035 partially formed of a transparent portion in the automatic analyzing apparatus 1 according to the modification. By configuring a part of the sample container 2035 with the transparent portion 360 in this manner, the strength of the sample container 2035 can be improved.

In both the examples of FIGS. 20 and 21, the liquid level height of the sample contained in the sample container 2035 can be acquired via the transparent portion 360 by performing image analysis on the imaged image data of the sample container 2035. In other words, the transparent portion 360 has such transparency that the height of the sample can be acquired by image analysis of the image data. For this reason, the sample container 2035 can also be configured by a sample cup for storing a small amount of sample, a glass bottle having high transparency, or the like.

According to at least one embodiment described above, the piercing arm can be normally lowered while reducing the burden on the user, and the overall throughput of the automatic analyzing apparatus 1 can be improved.

Note that the word “processor” used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the memory 8. Note that programs may be configured to be directly integrated in the processor instead of being storing in the memory 8. In this case, the processor realizes functions by reading and executing programs stored in the circuitry. Note that the processor is not limited to the case arranged as a single processor circuit, but may be configured as a single processor by combining a plurality of independent circuits to realize functions. Furthermore, a plurality of component elements in FIG. 1 may be integrated into one processor to realize the functions.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. An automatic analyzing apparatus, comprising:
a first imager configured to image a sample container in which a sample is contained and whose upper surface is sealed with a cap; and
processing circuitry configured to:
acquire a liquid level height of the sample contained in the sample container based on first image data which is image data of the sample container imaged by the first imager; and
determine, based on the liquid level height, a first parameter related to a descent operation of a piercing arm, and control, based on the first parameter, the descent operation of the piercing arm, the piercing arm holding a piercer needle for piercing the cap of the sample container.

2. The automatic analyzing apparatus according to claim 1, wherein the first parameter includes at least a descending amount of the piercing arm.

3. The automatic analyzing apparatus according to claim 1, wherein the first imager images the sample container after the sample container gets under the control of the automatic analyzing apparatus and before the piercing arm starts the descent operation.

4. The automatic analyzing apparatus according to claim 1, further comprising a second imager configured to image the cap of the sample container, a distal end portion of the piercer needle, and the sample contained in the sample container around the distal end portion, when the piercer needle pierces the cap of the sample container,
wherein the processing circuitry is further configured to determine whether or not the piercer needle normally penetrates the cap of the sample container based on second image data which is image data imaged by the second imager.

5. The automatic analyzing apparatus according to claim 4, wherein the processing circuitry is further configured to output a warning when it is determined that the piercer needle does not normally penetrate the cap of the sample container.

6. The automatic analyzing apparatus according to claim 4, further comprising an image memory configured to store the second image data when it is determined that the piercer needle does not normally penetrate the cap of the sample container.

7. The automatic analyzing apparatus according to claim 4, wherein the processing circuitry is further configured to transmit the second image data to the outside of the automatic analyzing apparatus when it is determined that the piercer needle does not normally penetrate the cap of the sample container.

8. The automatic analyzing apparatus according to claim 1, wherein
the automatic analyzing apparatus is connected to an external conveyance apparatus that is provided to the outside of the automatic analyzing apparatus and conveys the sample container to the automatic analyzing apparatus, and
the first imager images the sample container while the sample container is being conveyed by the external conveyance apparatus.

9. The automatic analyzing apparatus according to claim 1, wherein the sample container is held by a rack and stored in a rack sampler, or the sample container is stored in a disk sampler.

10. The automatic analyzing apparatus according to claim 1, wherein the processing circuitry is further configured to acquire information related to a rack on which the sample container is held from data other than the first image data.

11. The automatic analyzing apparatus according to claim 1, wherein the processing circuitry is further configured to determine, based on the liquid level height, a second parameter related to the descent operation of a sampling arm, and control, based on the second parameter, the descent operation of the sampling arm, the sampling arm holding a sampling probe that sucks the sample.

12. The automatic analyzing apparatus according to claim 1, wherein the processing circuitry is further configured to:
determine whether or not a type of the sample container can be specified based on the first image data; and
acquire, when the type of the sample container cannot be specified, the liquid level height of the sample contained in the sample container based on the first image data.

13. The automatic analyzing apparatus according to claim 12, wherein the processing circuitry is further configured to, when the type of the sample container can be specified, determine, based on the type of the sample container, the first parameter, and control, based on the first parameter, the descent operation of the piercing arm.

14. The automatic analyzing apparatus according to claim 1, wherein the sample container includes a transparent portion at least partially formed, and in the first image data, the liquid level height of the sample contained in the sample container can be acquired via the transparent portion.

15. A control method of an automatic analyzing apparatus comprising:
imaging a sample container in which a sample is contained and whose upper surface is sealed with a cap;
acquiring a liquid level height of the sample contained in the sample container based on first image data which is image data of the sample container; and
determining, based on the liquid level height, a first parameter related to a descent operation of a piercing arm, and controlling, based on the first parameter, the descent operation of the piercing arm, the piercing arm holding a piercer needle for piercing the cap of the sample container.
